(19) United States
(12) Patent Application Publication
(10) Pub. No.: US 2010/0010269 A1
(43) Pub. Date: Jan. 14, 2010

(54) APPARATUS AND PROCESS FOR USE IN THREE-PHASE CATALYTIC REACTIONS

(75) Inventor: Stanislaw Tadeusz Kolaczkowski, Bath (GB)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(73) Assignee: The University of Bath, Bath (GB)

(21) Appl. No.: 12/444,239

(22) PCT Filed: Oct. 5, 2007
(86) PCT No.: PCT/GB2007/003794

§ 371 Date: Jun. 12, 2009

(30) Foreign Application Priority Data
Oct. 6, 2006 (GB) 0619835.2

(51) Int. Cl. C07C 45/29; B01J 8/04

(52) U.S. Cl. 568/431; 422/190

(57) ABSTRACT

A reactor for carrying out a heterogeneously catalyzed reaction includes at least first and second reaction zones that are arranged in series and that each include catalytic material, heat transfer zones that are located between said serially arranged reaction zones, and a pulse-generating device, which is arranged to deliver pulses to liquid in the reactor. The reactor allows three-phase reactions to be carried out efficiently and can reduce the impact of deposited reaction by-products on reaction efficiency.

The invention relates to methods and apparatus for carrying out three-phase catalytic reactions. In particular, but not exclusively, the invention relates to methods and apparatus for carrying out three-phase catalytic oxidation or partial oxidation reactions, especially, for example, for manufacturing pharmaceutical agents, additives for pharmaceutical use, fine chemicals, and intermediates for use in the manufacture of the aforesaid. Other types of reaction to which the invention is applicable include, for example, hydrogenation reactions.

Within the pharmaceutical and fine chemicals industries, there are many examples of the production of chemical products and intermediates by the partial oxidation of organic liquids using strong oxidising agents such as nitric acid.

In the literature, there is evidence that more environmentally benign oxidants such as gaseous oxygen in combination with heterogeneous catalysts may be used to promote such reactions. In experiments described in Bavykin et al (Applied Catalysis A: General 288 (2005) 175-184), a compact multifunctional reactor is described. To demonstrate the viability of the reactor to be used for the partial oxidation of organic liquids, the oxidation of benzyl alcohol to benzaldehyde was studied as a model reaction. The benzyl alcohol was fed into the reactor in a solvent. The reactor consisted of parallel packed catalytic beds (channels varying from 2 to 5 mm in hydraulic diameter, using ruthenium (III) hydrated oxide catalyst supported on alumina of particle size approximately 150 micron), with a static mixer section at the inlet to each channel (for oxygen and liquid mixing), and neighbouring channels that contained a heat transfer fluid for maintaining a relatively uniform temperature along the length of the reacting zone. To sustain the reaction and maintain selectivity, the reactor was operated at a temperature of about 115° C. To facilitate the transfer of oxygen from the gas to the liquid phase, a pressure in the region of 8 bar was maintained in the reactor. The advantage of staged injection of oxygen along the reacting length was demonstrated, and two parallel channels were operated with a maximum catalytic bed length of 200 mm. It has now been found, in further experiments carried out in a larger-scale pilot reactor, that a number of problems are encountered using such an arrangement:
(1) Within one hour of operation, the pressure drop across the reactor started to increase, and after 2 hours of operation was at an unacceptable level. This had a consequential effect of reducing liquid flow in the reactor.
(2) When attempting to flush the reactor to remove any deposit that may have blocked the channels, the pressure drop increased further and liquid flow was restricted to an extent such that the flushing operation had to be stopped.
(3) Packing of the multiple channels with powdered catalyst, and then, after use, emptying and replacing such catalysts is very labour-intensive.
(4) Providing for and monitoring uniform distribution of gas and liquid across the channels is difficult. With the low linear velocities in the bed, gas/liquid maldistribution was potentially a contributing factor to the physical/chemical conditions that caused the pressure drop to increase across the bed.

In the experimental reactor described, it is believed that the pressure drop increase arose as a result of the deposition of by-products (e.g. benzoic acid) from the partial oxidation of benzyl alcohol to benzaldehyde in the packed reacting unit. For example, formation of deposits between the very small catalyst particles (150 micron) would soon start to restrict the flow through the bed, and hence cause an increase in pressure drop. It is postulated that the deposition occurred in the packed catalytic channels at locations where the liquid vaporised, or liquid did not adequately wet the surface of the particles, leading to the build up of solid/viscous layers that restricted the flow. This deposit build up would have also occurred on the glass fibre wool that supported the catalyst particles at the base of the individual channels, and on the glass wool at the top of the catalyst bed (retaining the catalyst in the packed zone in the channel). It is also possible that deposit build-up occurred in the static gas/liquid mixing channels. Likewise, if the target end product of the partial oxidation had been benzoic acid (for which the reaction conditions would need to be adapted accordingly), as larger quantities of benzoic acid would have been formed in the catalytic channels, difficulties would have been encountered a lot earlier in the operation.

A wide range of reactions results in target compounds that may form deposits and/or viscous residues if the liquid that acts as the solvent is vaporised. Likewise such deposits and/or residues may occur if, at the operating conditions in the reactor, non-volatile intermediates or by-products are formed.

GB 839066 describes a reaction apparatus in which liquid and gaseous reactants are caused to flow through, and react in, a fixed bed of solid catalyst, and in which the linear velocity of flow of the liquid reagent in the reactor is subjected to periodic and alternate increases and decreases by a superimposed force. The reaction apparatus includes an externally provided heat exchanger for heating one of the reactants before entry to the reactor and cooling the reaction products, and a cooler A for cooling the product mixture before separation of hydrogen for recycling.

There remains a need for an apparatus and method which allows in an effective manner the manufacture of compounds in three-phase catalytic reaction systems.

The invention provides a reactor for carrying out a heterogeneously catalysed reaction of at least one gaseous reactant and at least one liquid phase reactant comprising:

first and second discrete reaction zones arranged in series and each comprising catalytic material;

at least one heat transfer zone located between said first and second reaction zones for transfer of heat into, or away from, the reactor contents;

at least one inlet upstream of said first reaction zone for introduction of reactants;

at least one outlet downstream of said second reaction zone for egress of reaction product; and

a pulse-generating device, which is arranged to deliver pulses to fluid in the reactor.

In accordance with the invention, the reactor has a device for generating pulses.

The term “pulse” is used herein to mean the temporary movement of a volume of liquid in a forward and then reverse direction, also known as “oscillating the fluid”. Flow conditions in which repeated pulses are applied to the volume of liquid are known as “oscillatory fluid flow” or “pulsatile flow”. Methods of generating pulsatile flow are described in GB 839066, in EP 1076597A (where it is called “oscillating the liquid”), and also in EP 0540180A1 (where it is described as “pulsatile flow” and “oscillatory fluid flow”).

The generally oscillatory motion of the fluid as it passes through the catalytic material in the reaction zone causes agitation of the fluid leading to excellent mixing, efficient wetting of the catalyst, and especially to an advantageous washing effect in which deposits or viscous layers are prevented from forming on, or are washed away from, the surfaces of the catalytic material.

In the continuous polymerisation reactions described in EP 0540180A and EP 1076597A the catalytic substance is mobile and not confined to defined zones. It has been found that, surprisingly, a pulsing motion gives particular advantages in the case of a catalytic reactor having defined reaction zones and at least one intervening heat transfer zone according to the invention, and it is possible to achieve efficient catalysis notwithstanding the relative immobility of the catalyst and therefore more localised heat generation.

The pulses may be generated by any suitable method. Illustrative non-limiting examples of such methods are the movement of a piston, a diaphragm, or bellows. It has been found that the use of pulses, in accordance with the invention, leads to an advantageous increase in the efficiency of the catalytic reaction and/or in the lifetime of the catalyst before regeneration or other maintenance is required. It is believed that the increased efficiency and/or lifetime are attributable to one or more of the following:
(a) The pulsing action promotes mixing of gas and liquid in zones upstream/downstream of respective reaction zones, thereby dispersing the gas in a form of very fine bubbles throughout the liquid phase, increasing the surface area for gas-to-liquid mass transfer of gas, and the movement of fluid as a result of the pulse also promotes mass transfer of gas from the gas phase into the liquid phase.
(b) The pulsing action causes displacement of the liquid inside the reaction zones at a greater linear velocity than that which would arise if no pulse was generated. This in turn increases rates of mass transfer of reactants and products to and from the catalytic surface, and the transfer of heat to and from the catalytic surface.
(c) The displacement of liquid at a higher velocity in the reaction zones has a very important washing action on the surface of the catalyst. Should a deposit or viscous layer have been formed on the surface, which is soluble in the liquid phase, then the pulsing action on the fluid and subsequent liquid movement in the channels exhibits a washing effect, which cleans the catalyst surface. This in turn reduces the rate at which pressure drop would build up across the reactor.
(d) The combination of the pulsing action, with appropriate construction of the reaction zone, ensures that the possibility of having regions in the reaction zone that are not adequately flushed with fluid is minimized. This in turn reduces the chance of deposits building up which would increase the pressure drop across the reactor.
(e) At the inlet and outlet of the reaction zones, as the reactor is pulsed, the fluid is displaced in and out of the reaction zones promoting mixing with fluid in adjacent (for example, void) zones. This in turn has a beneficial effect on reducing axial temperature gradients in the catalytic section. The inlet and outlet faces of the catalytic sections act as baffles that in combination with the pulsating action promote the mixing process taking place in the adjacent void zones.
(f) The pulsating action acting to move the liquid relative to the inlet and outlet faces of the heat transfer zone(s) helps to promote gas-liquid mixing, especially in void zones advantageously provided to accommodate and promote reactant mixing.
(g) At the end of a reaction, the contents of the reactor can be displaced with a wash fluid, and the pulsating action activated (with or without the addition of a gas) to promote internal cleaning and the washing from the catalytic surface of any deposits that may have been formed. This aspect is particularly important for a pharmaceutical application.

The invention is of particular application to heterogeneously catalysed gas-liquid reactions where either one or more components in a liquid feed to the reactor, or product(s) of the reaction (desirable or undesirable) are relatively non-volatile (that is, would form a viscous layer or deposit if the liquid in which they were contained was vaporised) at the reaction conditions in the catalyst zone (temperature/pressure), but they are soluble/mobile if washed with the liquid phase.

Advantageously, there are one or more further reaction zones downstream of said second reaction zone. In one embodiment of the invention, between successive reaction zones, there is located a void zone in which mixing can take place. In another embodiment of the invention, between successive reaction zones, there is a transfer conduit, the reaction zones being housed in separate vessels.

Between the first and second reaction zones, and where there is one or more additional reaction zones then preferably in each case between successive reaction zones, there is at least one heat transfer zone.

The heat transfer zone or zones are arranged to allow transfer of thermal energy into or out of the reactant fluids. In a preferred arrangement, the or each heat transfer zone comprises a heat transfer structure which is located within the said heat transfer zone, and is advantageously in intimate contact with the process fluid. The heat transfer structure is advantageously in thermal communication with a heat sink and/or a heat source. The heat sink or heat source is preferably located externally of the reactor vessel. Preferably, the heat transfer structure defines one or more enclosed channels in which heat transfer fluid can flow in isolation from, but in heat-exchange relationship with, the fluid in the reactor. The heat transfer structure may, instead, comprise compact heat exchanger plates.

The preferred alternating arrangement of reaction zones and heat transfer zones provides good temperature control within the reactor, allowing, for example, the removal of excess heat from reactant mixture that has increased in temperature on passing through the preceding reaction zone.

The catalytic material may be particulate material, for example, with a hydraulic diameter of from 2 to 10 mm. The catalytic material may comprise a matrix structure defining substantially parallel channels that extend through the reaction zone. The or each reaction zone advantageously has a monolithic structure comprising an inert matrix support upon which catalyst is supported, for example, having channels with a hydraulic diameter of from 1 to 5 mm.

In the methods of EP 0540180A and EP 1076597A, baffles are provided in the apparatus described and apparently contribute to obtaining the desired outcome from the pulsatile flow described. The inlet sections to the heat transfer zones may be arranged to act as baffles, and, for example, the cross-sectional area of the vessel taken up by those baffles can be from 25 to 75%. Likewise, depending on the choice of catalyst support (e.g. pellets, monolith, other structured supports), as the cross-sectional area of the vessel taken up by the catalytic structure could vary, for example, from 20 to 60%, the inlet sections to the catalytic zones may also exhibit some of the beneficial characteristics of baffles.

Advantageously, said at least one inlet comprises a first inlet for admission of liquid reactant, and a second, gas injection inlet for introducing a gas into the inlet zone. The gas may consist of, or include, a gaseous reactant, for example, oxygen.

Advantageously, the pulse-generating device is able to generate pulses at more than one frequency. The frequency may be adjustable continuously or may be adjustable stepwise. The frequency may be adjustable between two or more predetermined frequencies.

Advantageously, the pulse-generating device is able to generate pulses by means of displacing a volume of liquid, which volume is advantageously adjustable. The amplitude of the pulse, for example, the displacement volume, may be adjustable continuously or in stepwise manner. The amplitude, for example, displacement volume, may be adjustable between two or more predetermined amplitudes. In practice, the pulse-generating device is set at a frequency and set at an amplitude each selected to effect, in combination, the desired outcome in the internal geometry of the reactor. The pulse is advantageously generated at a single location in the reactor or, for example, simultaneously, sequentially, or out of sequence at a number of locations, in which case more than one pulse generator may be provided. What is important is the effect that the pulse has on the overall performance of the reactor. The selection of effective frequencies and amplitudes for a given reactor may be accomplished by routine experimentation. By way of illustration, the frequency may be, for example, up to 10 Hz, advantageously from 0.5 to 10 Hz, especially 0.5 to 7 Hz. The amplitude is significantly dependent upon the reactor dimensions and the desired amplitude of the oscillation in the vessel. The amplitude (or effective longitudinal movement of fluid in the vessel as the pulse is applied), for example, may vary from 0.025 to 0.5 times, especially 0.025 to 0.4 times, or 0.05 to 0.5 times, especially 0.05 to 0.4 times, the internal hydraulic diameter of the vessel. In practice, however, the reactor will preferably be arranged such that the amplitude will be of a desired value, for example, from 2 to 30 mm within at least one, and preferably each, reaction zone. It is also possible for the amplitude to be higher if desired, for example, 1 to 15 cm. It will be appreciated that a first oscillation amplitude, within a reaction zone that is generally packed relatively densely with catalyst, may be associated with a second, lower amplitude in intervening zones, especially mixing zones, which may have a higher void fraction, or free cross-sectional area for liquid flow, than the reaction zone. The calculation of a suitable amplitude within a wholly void length of the vessel required to generate a desired amplitude within a reaction zone will be a routine matter for those skilled in the art based on known or readily determinable parameters including the free void fraction in the reaction zone

The amplitude and frequency of pulsation may need to be adjusted for different reactions in the same size of reactor. For example, if processing a fluid where the temperature rise for the reaction is expected to be relatively high, then by increasing the amplitude of the pulse, it may be possible to achieve the necessary temperature control across the catalyst section. Advantageously, the reactor comprises from 2 to 15 zones arranged in series.

In preferred embodiments, there is a multiplicity of reaction zones, with intervening heat transfer zones. This is particularly important when good temperature control needs to be maintained, so as to maintain the selectivity for the reaction. As partial oxidation reactions are exothermic, if the fluid is not cooled, then as the reaction proceeds the temperature of the fluid would increase. This in turn could lead to temperatures in the catalytic zones at which side reactions (undesirable) would start to become more significant, and the selectivity of the desired reaction would be affected. So reaction zones, with intervening heat transfer zones, enable good temperature control to be achieved. A typical reactor for certain pharmaceutical applications may have, for example, five reaction zones arranged one above the other, an inlet zone including an inlet for reactant beneath the lowermost reaction zone, seven heat transfer zones, including a respective heat transfer zone between each pair of adjacent reaction zones as well as upstream of the first reaction zone and downstream of the last reaction zone, void zones between successive reaction zones, with a further inlet for a gas in each of said void zones as well as in the inlet zone and in an outlet zone downstream of the last reaction zone. In certain preferred embodiments the or each reaction zone has a length, in the general direction of travel of the reactants through the reactor, of from 10 to 200 mm, preferably 30 mm to 80 mm. Greater or smaller lengths may also be appropriate in some cases, depending on the application and operating characteristics of the pulse generator (frequency and amplitude of operation).

The invention also provides a method of continuously effecting a catalytic reaction, comprising passing a reaction mixture in sequence through a first reaction zone comprising catalytic material, a heat transfer zone, and a second reaction zone comprising catalytic material, and applying a pulsing motion to the reaction mixture for agitation of the reaction mixture. Agitation of the mixture using pulses in that manner is believed to result in an advantageous mixing and washing effect on the catalyst surface, enhancing working life thereof.

Advantageously, the reaction mixture is caused to pass in series through a multiplicity of reaction zones and intervening heat transfer zones and void zones for mixing. Advantageously, the pulsing motion is imparted by displacement of a volume of liquid.

In an especially preferred method of the invention, the catalytic reaction is oxidation or partial oxidation of an organic compound. One preferred reaction product of the invention is a pharmaceutical agent or an intermediate for use in the manufacture of a pharmaceutical agent. Certain embodiments of the invention will be described in detail below, by way of illustration only, with reference to the accompanying drawings, in which:

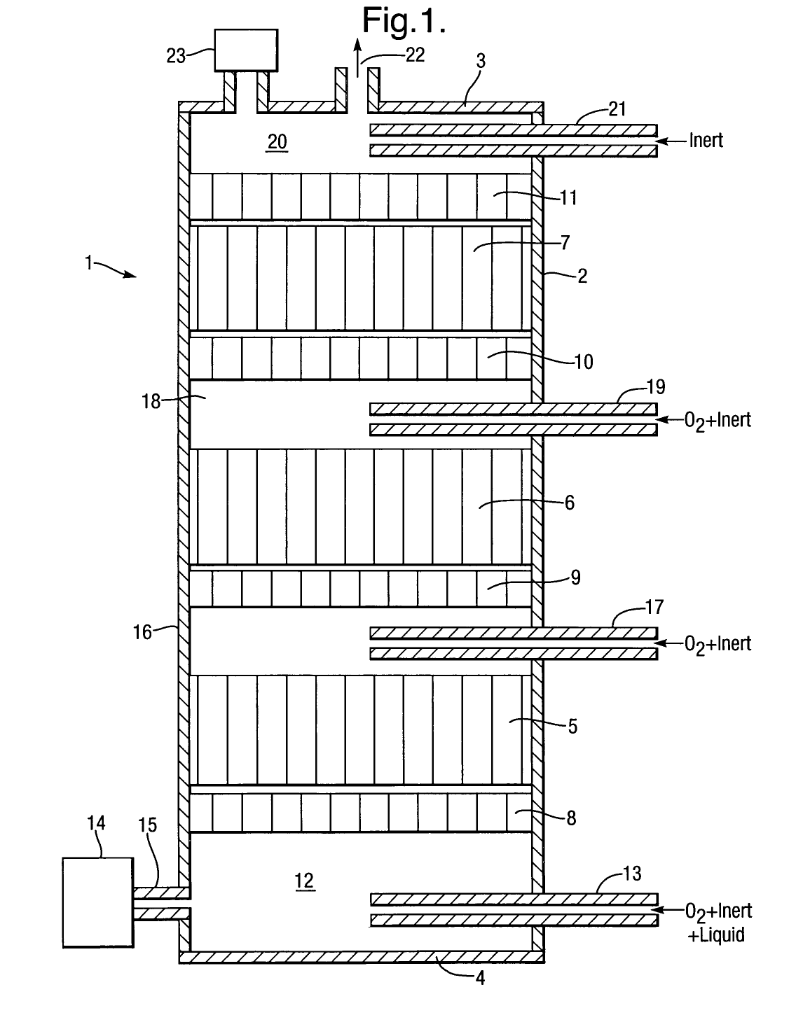

FIG. 1 is a section through an apparatus according to a first embodiment of the invention;

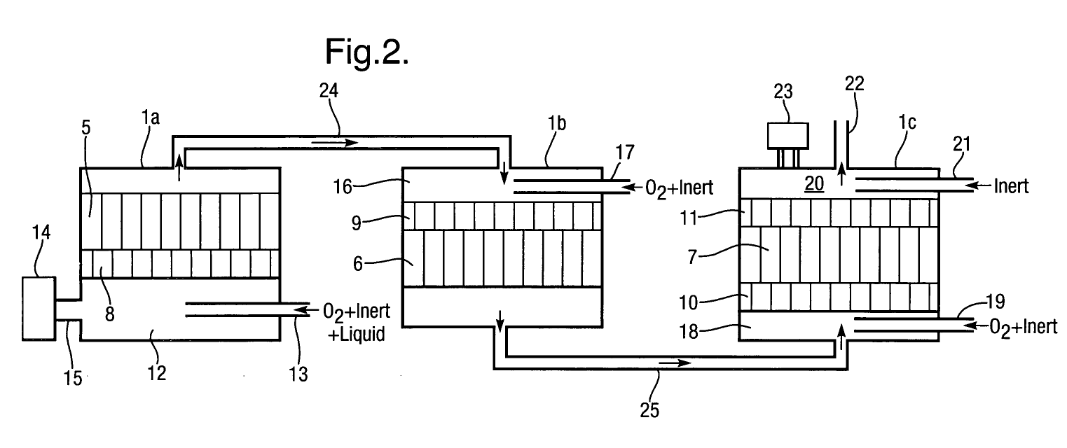

FIG. 2 is a section through an apparatus according to a second embodiment of the invention;

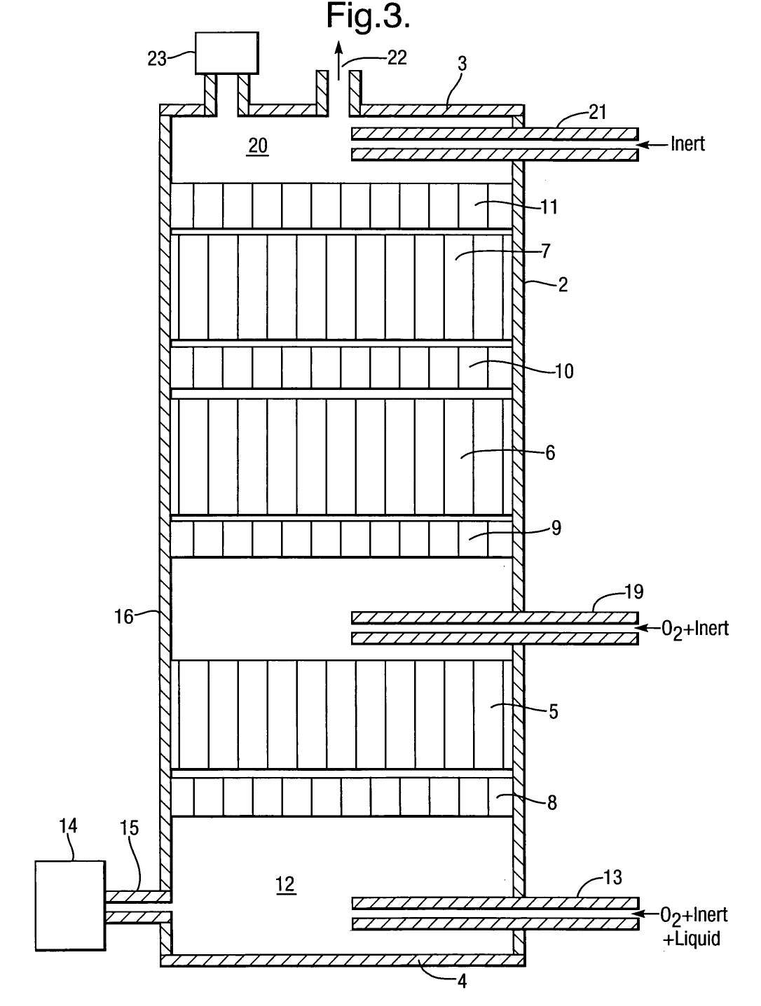

FIG. 3 is a section through an apparatus according to a third embodiment of the invention.

With reference to FIG. 1, there is shown a three-bed reactor 1. The reactor 1 is illustrative of a reactor suitable for use in a catalytic oxidation process for the manufacture of chemical compounds, for example, intermediate compounds for use in making a pharmaceutical agent using gaseous oxygen, but for simplicity is shown with only three reaction zones or beds, whilst in practice in such an application a higher number of beds, for example five or six, is preferred.

The reactor 1 consists of a vessel having a circumferential wall 2, a downstream end wall 3, and an upstream end wall 4. In FIG. 1, the vessel is mounted in a vertical orientation with upstream end wall 4 at the bottom and downstream end wall 3 at the top, but other orientations are also possible. As will be apparent from the description below, the reaction fluids flow upwardly through the vessel 1, and for the avoidance of doubt the words “upstream” and “downstream” in relation to the vessel or components thereof are to be understood as relating to the general direction of travel of fluid in the reactor so that, in the case of the reactor in FIG. 1, the upstream extremity of the vessel or any component is positioned at the bottom of the vessel or component respectively whilst the downstream extremity of the vessel or any component is positioned at the top of the vessel or component respectively. The vessel may have any suitable cross-sectional shapes e.g. circular, square. In use, the inside surfaces of the walls 2, 3, 4 of the vessel are maintained at a desired operating temperature, for example a value between 50 to 200° C., the desired temperature depending on the chemistry of the chosen oxidation reaction. For that purpose, at least the wall 4 and optionally the walls 2 and 3 are provided with suitable temperature control means (not shown), which may be of any suitable kind, for example, external electrical heating, or heating jackets or tubes containing heat transfer fluids.

Inside the vessel, as illustrated in FIG. 1, are three reaction zones 5, 6, 7 through which, in use, the gas and liquid can flow, and where the catalytically supported chemical reactions take place. Also present are heat transfer zones 8, 9, 10 and 11, through which the gas and liquid can flow, and where the temperature of the fluid between and in the reaction sections is controlled, for example avoiding temperature increases with a value between 3 to 20° C. above the desired operating temperature. As is apparent from FIG. 1, the heat transfer zone 8 is immediately upstream of the first reaction zone 5, the heat transfer zone 9 is immediately upstream of the reaction zone 6, and there are two heat transfer zones 10 and 11, immediately upstream and downstream, respectively, of the reaction zone 7.

Beneath the heat transfer zone 8 is an inlet zone 12. A feed nozzle 13 is provided in a lower region of the inlet zone 12, for the introduction of the reactant materials, for example a solution of a first reactant material in a solvent and, as second reactant, gaseous oxygen. The inlet zone 12 serves for mixing of the reactants (if required) and for dispersion of gaseous reactant into fine bubbles. A pulse-generating device 14 is provided at the wall 4 communicating via conduit 15 with the inlet zone 12. The nature and function of the pulse-generating device are described in more detail further below. The pulse-generating device may, if desired, be positioned beneath the bottom wall 4 instead of in the position shown in FIG. 1.

Between the reaction zone 5 and the heat transfer zone 9 is a void zone in the form of mixing zone 16, which is provided with a gas injection nozzle 17 for injection of gas, for example oxygen in admixture with an inert carrier gas such as nitrogen. A second void zone in the form of second mixing zone 18 is provided between the reaction zone 6 and the heat transfer zone 10, which as in the case of the mixing zone 16 is provided with a gas injection nozzle 19 for introduction of further reactant gas optionally with inert carrier gas. Adding the oxygen in a staged manner (e.g. via nozzles 13, 17, 19), rather than a single addition (e.g. via nozzle 13) may be beneficial in some applications. For example, for the partial oxidation of benzyl alcohol, it has been shown to result in higher overall rates of reaction. In addition, the maximum rate of reaction in individual catalytic zones could be controlled by the amount of oxygen in the fluid at the point of entry to a particular catalytic zone, and hence the amount added via a nozzle at the entry to that zone. An outlet zone 20, above the heat transfer zone 11, also includes a gas injection nozzle, in the form of nozzle 21, which is arranged to introduce an inert gas into the outlet zone, should that be required for safety reasons to reduce the concentration of oxygen in the effluent from the reactor. The outlet zone provides a mixing region where gas is introduced through the nozzle 21. An effluent port 22 is provided for removal of the product from the reactor for subsequent working up and purification of the target compound. Whilst in FIG. 1 22 is shown as a single port it may instead consist of multiple ports.

The mixing zones 16 and 18 allow space for mixing, and this is particularly important when additional gas (e.g. oxygen) is injected in between the reaction zones. This creates a free zone (in the cross-sectional plane, and short axial distance) in which the gas may be dispersed into fine bubbles, and for it also to be adequately mixed across the cross-section of the reactor. However, depending on the design of the reactor, free space may not be necessary between all of the catalyst and heat transfer sections.

The pulse-generating device 14 includes a device (not shown) for displacing a volume of liquid through the channel 15 into the inlet zone 12. The device may be in the form of, for example, a diaphragm, bellows or a piston, which reciprocates between first and second positions. The volume of liquid expelled from the channel 15 into the inlet zone 12 on a first reciprocatory stroke will in most circumstances be substantially equal to the volume of liquid withdrawn into the channel 15 from inlet zone 12 on the opposite reciprocatory stroke of the device (the displacement volume). It is thought that the propulsion of the displacement volume into the inlet zone 12 causes a general flow of the liquid and entrained fine bubbles of gas upwards. The subsequent return of the displacement volume into the channel 15 on reversal of the pulse-generating device then results in a general reversal of flow in the vessel. Repeated pulses thus cause the liquid to tend to move first upwards into each of the reaction zones and then downwards again. As a result of repeated pulsing at relatively short intervals (for example, a plurality of pulses per second), the motion of the liquid imposed by the repeated pulses and by interaction of the moving liquid with the obstacles in its path is complex, and is believed to enhance mixing and washing of the catalyst surface.

The reactor zones 5, 6, 7 include catalytic material in any suitable form, for example, in particulate form or in the form of a unitary body defining pathways for the reactant gas/liquid mixture. Thus, in one preferred embodiment, the reactor zones each comprise a bed of particulate material, usually comprising catalytic material supported on an inert support such as carbon, alumina or silica. For example, the particulate material may be pellets comprising the catalyst, the pellets preferably being of dimensions selected to give a hydraulic diameter of from 2 to 10 mm. In another, especially preferred embodiment, the reaction zone comprises a structured catalyst bed in the form of a monolith with parallel channels, preferably with a hydraulic diameter of from 1 to 5 mm. Preferably, the monolith comprises catalyst material supported on an inert support matrix.

Heat transfer zones 8, 9, 10 and 11 may be of any suitable structure having regard to their function of helping to maintain the temperature of the contents of the reactor within desired limits. Suitable structures include one or more members located within the reactor interior and in contact with the fluid in the heat transfer zone, those members being arranged to supply heat to, or withdraw heat from, the fluid. In general, the heat transfer zones define enclosed channels in which heat transfer fluid can flow in isolation from, but in heat-exchange relationship with, the process fluid in the reactor. The heat transfer sections can also serve as a baffle to promote gas/liquid mixing as the contents of the vessel are pulsed. Suitable heat transfer devices for use in the heat transfer zones include, for example, tubes, or compact heat exchanger types of plates.

Various modifications of the apparatus of FIG. 1 are possible. Below are described a number of illustrative examples of such variations.

In FIG. 1, the gas and liquid are shown to be fed into the inlet zone 12 via a single nozzle 13. If desired, the gas and liquid could be fed separately into the section 12, from the side, from the base, or both, and optionally via a suitable distribution device to promote gas/liquid mixing/distribution.

Likewise, one or more, and preferably all, of nozzles 17, 19 and 21 for the addition of gases may have a distribution device for improving gas/liquid distribution and mixing. In nozzles 13, 17 and 19, an inert gas (e.g. nitrogen) may be added with the oxygen for safety reasons—so as to dilute the gas phase concentration of oxygen. Also, during start-up of the reactor, at shut-down, or during cleaning, it may be preferable to operate with the injection through one or all of 13, 17, 19 and 21 of just an inert gas without the addition of oxygen.

In the inlet zone 12 and mixing zones 16, 18 and 20, the injection point of the reactants and/or inert gases could take place above or below the heat transfer zone.

In some modes of operation it may be desirable or necessary to include a pressure damper 23 in the apparatus for relieving any build-up of excessive pressure that might otherwise damage the reactor or its components. For example, the provision of a damper may be advantageous where the reactor may operate at very low gas/liquid ratios. The pressure damper could be situated near the outlet 22 of the reactor, and would allow the liquid level in the vessel to rise and fall in response to the combination of pressure pulses generated by device 14, and the gas/liquid ratio in the vessel. Device 23 could operate in a variety of ways, for example, it could be simple and contain a volume of gas that is compressed, or the liquid could be separated from the gas by a movable barrier, such that when it moved, it compressed gas on the other side of the barrier. The pressure damper could also be located at other places on the vessel. The movement of liquid volume allowed or set on damper 23 would depend on the settings selected for the pulse-generating device 14, and gas/liquid ratios in the vessel. Another suitable damping means is, for example, that shown in EP 0540180A in which the pulse-generator allows for displacement of liquid in the outlet to compensate for the displacement of liquid near the inlet of the reactor.

The size of the reactor and its components may be selected according to the application. In the partial oxidation application described, for a liquid feed rate of 4.2 kg per hour, consisting of a 10% benzyl alcohol feed in a solvent, then as an example, the overall length of the reactor would be 1000 mm, with an internal diameter of 90 mm, consisting of seven catalytic zones, each catalytic zone being 70 mm long and consisting of a Pt catalyst distributed on a carbon support in the form of a monolith. There would also be eight heat exchange zones, seven oxygen injection ports, one liquid feed inlet port and one product outlet port. The operating temperature would be 110° C., and the operating pressure 15 Bar (1.5×10⁶ Pa). The apparatus may, however, be significantly larger or significantly smaller, depending on the application, with appropriate adaptation of the structure described, such appropriate adaptation being a matter of routine workshop modification for those skilled in the art.

The length of the catalytic sections 5, 6 and 7 could be varied for any given reactor, and this would depend on the temperature rise expected over the respective section of catalyst, and the temperature control required to sustain the selectivity of the chemical reaction. For example, if the temperature was not to rise by more than 10° C., then the length of the catalyst section would be selected to ensure that the conversion across the catalyst length does not result in a release of energy that would exceed this temperature. The zones 5, 6, 7 normally contain the catalyst supported on a suitable support to effect the desired reaction. If the exotherm were such that a short catalytic section was required in the space provided to avoid generation of unacceptable temperature increases (for example, in reaction zone 5 a 20 mm length instead of a 30 mm section), then a 10 mm inert section of catalyst support material could be added to ensure that the overall length of 30 mm was preserved and similar hydrodynamic conditions were maintained.

The apparatus is constructed to allow access to the reaction zones as and when required for maintenance including replacement of spent catalytic material. Access may be permitted using any suitable structure. In one suitable arrangement, the vessel is constructed to allow access along the central axis of the vessel, for example, by opening one or more of a number of bolted flanged sections along the axis of the vessel. In another suitable arrangement, there may be provided one or more removable side plates which, in use, are sealed to the adjacent portions of the vessel, and which can be unsealed and withdrawn in the horizontal direction to allow lateral access into the interior of the vessel.

In use, the catalytic oxidation reaction is carried out on a continuous basis with continuous introduction at feed nozzle 13 of liquid phase containing a first reactant and of gas, especially oxygen. The contents are maintained at an elevated pressure, for example at a value of between 5 to 30 bar. The contents of the reactor move upwards passing sequentially through the reaction zones 5, 6 and 7, and ultimately flowing out of the exit port 22, from which they are transported for working up, including removal of excess gas, extraction and purification of the reaction product, and recycling, if appropriate, of the recovered gas and solvent. During passage through the reactor, the liquid phase is subjected to repeated pulses which induce a generally oscillatory motion within the liquid, promoting dispersion of the gas into fine bubbles and otherwise improving mixing, as well as causing a washing effect of the liquid on the catalyst surfaces. The consequential improved mass transfer and effective washing of the catalytic surface allow the reaction to proceed more efficiently and the lifetime of the apparatus before shutdown for maintenance can be improved.

In a further embodiment shown in FIG. 2, the reactor consists of a separate number of vessels 1a, 1b, 1c that, in combination, perform the same function as the single vessel of FIG. 1. Use of separate vessels may be preferred in certain applications, to facilitate the removal of catalyst sections, and visual inspection of the interior of the reactor. Vessel 1a communicates with vessel 1b via conduit 24. Vessel 1b communicates with vessel 1c via conduit 25. Flow direction in the arrangement of FIG. 2 is upward, then downward, then upward. However, different combinations of upward and/or downward flow could be used. In FIG. 2, reference numerals have the same meaning as in FIG. 1.

In the embodiment of FIG. 3, where the reference numerals used are the same as those used in FIG. 1 they are to be understood as having the same meaning. In the embodiment of FIG. 3, however, the heat transfer section 10 is positioned between two reaction zones 6 and 7, there being no mixing zone and gas injection nozzle between those reaction zones. Optionally, as illustrated in FIG. 1, a further heat transfer zone 11 could be positioned as shown downstream of the reaction zone 7.

Other processes to which the reactor of the invention is suited include other catalytic liquid/gas reactions, for example, catalytic hydrogenation reactions.

These types of reactions are discussed extensively in the literature, and can be performed in two main categories of three-phase reactors. These are slurry reactors, in which the solid catalyst is suspended, and packed-bed reactors where the solid catalyst is fixed. Generally, (a) as the overall rate of reaction (in these types of reactors) is limited by mass transfer steps, and (b) heat transfer is also a key consideration to achieve good temperature control, then most of the features/phenomena described in the present specification (e.g. application of pulsatile flow conditions in combination with reaction zones and one or more heat transfer zones, and the way in which they work) would also be particularly suitable to effect hydrogenation reactions. The main differences would be that the gaseous stream acting as one of the reactants would be hydrogen (rather than oxygen as in the earlier description), and the liquid feed would be in the form of a hydrocarbon liquid phase which would contain the other reactant(s) either in a concentrated form, or they could be dissolved in a solvent (similar to partial oxidation application). Reaction conditions would depend on the chemical hydrogenation reaction implemented and choice of catalyst. Suitable catalysts are well described in the literature (e.g. see review article by: Cybulski et al, (2006) Monolith catalysts for three-phase processes, in Structured Catalysts and Reactors, Ed Cybulski and Moulijn, 2^{nd} Edition, Taylor & Francis, pgs. 355-391).

As hydrogenation reactions are endothermic, then during normal operations (i.e. not during start-up or shut-down) the heat transfer zones (e.g. in FIG. 1, zones: 8, 9, 10 and 11), would be used to add heat (rather than as in the partial oxidation application, which is exothermic, where heat is removed). Provision to add an inert gas such as nitrogen (e.g. via nozzles 13, 17, 19 and 21 in FIG. 1), either with hydrogen, or instead of hydrogen, may also be desirable for a variety of different operational reasons (especially start-up or shut-down). Likewise, the catalyst in the zones (e.g. FIG. 1, zones 5, 6 and 7), could be in a pellet, monolith or other structured form, although the preferred method is to use a monolith structure. The benefits that would arise from the washing action (described for the above-mentioned oxidation application) would clearly depend on the composition of the products and/or by-products formed. The dimensions of the reactor, and of the reaction and heat transfer zones would depend on the throughput for which the reactor was designed, and the lengths of these respective zones would depend on the level of temperature control that had to be maintained which would be influenced by the heat of the specific reaction implemented and the rates of reaction in their respective catalytic zones. The total overall length of the active catalytic zones would depend on the conversion that had to be achieved in the reactor.

In general, for both of the illustrative applications specifically described herein (hydrogenation and partial oxidation) the apparatus and method could be incorporated into a process that has product separation units positioned between reaction units (apparatus as described with reference to the drawings herein), or be operated with recycle stream(s). In addition, in accordance with normal custom and practice in the industry, provision would be made for an appropriate level of process control, instrumentation, and safety features, to satisfy a particular application or special requirements to match the operator's needs. It will be appreciated that additional and alternative features of structure and operation described above in relation to FIG. 1 may where appropriate analogously be applied to the reactors of FIGS. 2 and 3.

Typically conditions for use in the reactor of the invention would comprise a temperature in the range of 50 to 150° C. and pressure of 5 to 20 bar (0.5 to 2×10⁶ Pa) for a partial oxidation reaction and a temperature of 30 to 105° C. and pressure of 5 to 80 bar (0.5 to 8×10⁶ Pa) for a hydrogenation reaction.

1. A reactor for carrying out a heterogeneously catalyzed reaction of at least one gaseous reactant and at least one liquid phase reactant, the reactor comprising:
first and second discrete reaction zones arranged in series and each comprising catalytic material;
at least one heat transfer zone located between said first and second reaction zones for transfer of heat into, or away from, contents of the reactor;
at least one inlet upstream of said first reaction zone for introduction of reactants;
at least one outlet downstream of said second reaction zone for egress of reaction products; and
a pulse-generating device, which is arranged to deliver pulses to liquid in the reactor.

2. A reactor according to claim 1, further comprising one or more further reaction zones downstream of said second reaction zone.

3. A reactor vessel according to claim 1, wherein the reactor vessel includes in total from 4 to 15 reaction zones arranged in series.

4. A reactor according to claim 3, wherein the reactor vessel includes in total, five reaction zones arranged one above the other.

5. A reactor according to claim 1, further comprising a void zone in which mixing can take place, the void zone disposed between the first and second discrete reaction zones.

6. A reactor according to claim 5, further comprising a further inlet for at least one reactant provided in said void zone.

7. A reactor according to claim 1 further comprising a transfer conduit disposed between the first and second discrete reaction zones.

8. A reactor according to claim 2, further comprising at least one further heat transfer zone.

9. A reactor according to claim 8, in which a heat transfer zone is present between each successive pair of adjacent reaction zones.

10. A reactor according to claim 1, in which the at least one heat transfer zone comprises a heat exchange fluid in thermal communication with the fluid.

11. A reactor according to claim 1, in which the at least one heat transfer zone comprises a heat transfer device that occupies at least a proportion of the internal cross-section of the reactor.

12. A reactor according to claim 1 claims, in which the catalytic material is particulate material.

13. A reactor according to claim 12, in which the particulate material comprises particles having a hydraulic diameter of from 2 to 10 mm.

14. A reactor according to claim 1, in which the catalytic material comprises a matrix structure defining substantially parallel channels that extend through at least one reaction zone.

15. A reactor according to claim 14, in which the at least one reaction zone has a monolithic structure comprising an inert matrix support upon which catalyst is supported.

16. A reactor according to claim 14, in which the channels have a hydraulic diameter of from 1 to 5 mm.

17. A reactor according to claim 1, in which said at least one inlet comprises a first inlet for admission of liquid reactant and a second, gas injection inlet for introducing a gas into the inlet zone.

18. A reactor according to claim 1, in which the pulse-generating device applies pulses by means of displacing a displacement volume of liquid into the reactor.

19. A reactor according to claim 1, in which the pulse-generating device comprises at least one of a frequency adjusting device for adjusting the frequency of pulses or a pulse adjusting device for adjusting the amplitude of the pulse.

20. (canceled)

21. A reactor according to claim 1, in which the first and second discrete reaction zones each have a length (in the general direction of travel of the reactants through the reactor) of from 10 mm to 200 mm.

22. A method of continuously effecting a catalytic reaction, comprising:
passing a reaction mixture in sequence through a first reaction zone comprising catalytic material, a heat transfer zone, and a second reaction zone comprising catalytic material; and
applying a pulsing motion to the reaction mixture for agitation of the reaction mixture.

23. A method according to claim 22, wherein passing a reaction mixture comprises passing the reaction mixture in series through a plurality of reaction zones.

24. A method according to claim 22, in which the pulsing motion is imparted by displacing a volume of liquid.

25. A method according to claim 23, in which the heat transfer zone is arranged for transporting away heat generated in the reactor.

26. A method according to claim 22, in which the heat transfer zone can be used for supplying thermal energy to the reactor contents.

27. A method according to claim 22, in which the catalytic reaction is oxidation or partial oxidation of an organic compound.

28. A method according to claim 22, in which the reaction product is a pharmaceutical agent or an intermediate for use in the manufacture of a pharmaceutical agent.
